(19) United States
(12) Patent Application Publication
(10) Pub. No.: US 2007/0077306 A1
(43) Pub. Date: Apr. 5, 2007

(54) Apatite particle, method of producing the same, apatite particle-gene complex, and method of gene transfection

(76) Inventors: Toshihiro Akaike, Kanagawa (JP); Ezharul Hoque Chowdhury, Kanagawa (JP)

Correspondence Address: PATTERSON & SHERIDAN L.L.P., 595 SHREWSBURY AVE, STE 100, FIRST FLOOR, SHREWSBURY, NJ, 07702, US

(21) Appl. No.: 10/584,722

(22) PCT Filed: Dec. 27, 2004
(86) PCT No.: PCT/JP04/19549

§ 371 Date: Jul. 31, 2006

Related U.S. Application Data
(60) Provisional application No. 60/532,845, filed on Dec. 26, 2003.

(51) Int. Cl. A61K 48/00; C12N 15/87; A61K 9/14

(52) U.S. Cl. 424/489; 435/459; 977/906; 514/44

(57) ABSTRACT

When a solution of an inorganic phosphoric acid, added to with calcium ions and magnesium ions, is incubated for a preset time duration, an apatite particle obtained is reduced in size, depending on the concentration of the magnesium ions. By employing an apatite particle, for which the concentration of the magnesium ions and the incubation time duration are controlled adequately, it is possible to improve the gene transfection efficiency into the cells and the gene expression efficiency in the cells.

This application claims priority based on U.S. Preliminary Application No. 60/532,845, filed in United States of America on Dec. 26, 2003. By reference, this United States Application is incorporated into and becomes part of the present application.

### TECHNICAL FIELD

This invention relates to an apatite particle, that may be used with advantage for transfection of a gene into, for example, a cell, a method of producing the apatite particle, an apatite particle-gene complex, and a method of gene transfection.

### BACKGROUND ART

Gene transfection into a cell is a technique indispensable for analyses of the structure and functions of the gene or a control mechanism for the gene. This technique is similarly crucial for production of the therapeutically crucial protein on the industrial basis, gene therapy or for DNA vaccination.

Among the methods so far known for transfecting the gene into cell, there has so far been known a method consisting in incorporating the target gene into, for example, the viral DNA, to generate infected viruses, which are used for gene transfection. This method is extremely high in the transfection efficiency and hence is stirring up notice as an epoch-making method for gene therapy for a variety of genetic or acquired diseases. However, such gene transfer by the viral DNA has a serious drawback that, since the viruses non-specifically infect various sorts of cells, the genes may be transferred to other than the targeted cell. The probability is also high that the viral genome per se is incorporated into the chromosome, thus possibly leading to unforeseen side-effects in time to come. Hence, a non-viral system, which should take the place of the viral system, employing the viral vector, has been desired.

Among the pre-existing non-viral systems, there are known methods exploiting a synthesized lipid, such as liposome, a peptide, such as poly-L-lysine, a dendrimer, such as polyamide amine, other polymers, such as polyethylene imine, or calcium phosphate. Of these, the method employing calcium phosphate is based on a phenomenon in which an apatite particle, formed by inorganic phosphoric acid and calcium ions, forms a complex with DNA, and is co-precipitated with DNA, with the so formed apatite particle/DNA complex then being taken up by the cell by endocytosis. This method is commonly used for transfecting the gene into the cell. Reference is to be made to, for example, the Publication 1 “Fasbender, A. et al., 1998”, ‘Incorporation of Adenovirus in calcium phosphate precipitates enhances gene transfer to airway epithelia in vitro and in vivo’, J. Clin. Invest., 102, p. 184-193”, the Publication 2 “Toyoda, K. et al., 2000, ‘Calcium phosphate precipitates augment adenovirus-mediated gene transfer to blood vessels in vitro and in vivo’, Gene Ther., 7, p. 1284-1291” and to the Publication 3 “Urabe, M. et al., 2000, ‘DNA/calcium phosphate mixed with media are stable and maintain high transfection efficiency’, Anal. Biochem., 278, p. 91-92”.

Meanwhile, although this gene transferring method, employing calcium phosphate, is used nowadays as a routine method, it is low in gene transfer efficiency, which has proved a barrier in gene expression both in vitro and in vivo. The reason is possibly such that the apatite particle is increased in size, as the time of incubation of the inorganic phosphoric acid and the calcium ions is prolonged, with the result that uptake of the gene into the cell is lowered. Thus, in the Publication 4 “Jordan, M. et al., 1996 ‘Transfecting mammalian cells: optimization of critical parameters affecting calcium-phosphate precipitate formation’, Nucleic Acids Res., 24, p. 596-601”, there is disclosed a technique of shortening the incubation time for controlling the size of the apatite particle. However, this method has a drawback that it is difficult to apply for a case where much plasmid DNA is transferred at a time into cells and hence larger quantities of apatite particles are necessitated.

### DISCLOSURE OF THE INVENTION

In view of the above depicted problems of the related art, it is an object of the present invention to provide an apatite particle suppressed in particle size to a nano-order size and which is capable of transferring the gene highly efficiently into a cell to enable its expression in the cell, and a method of producing the apatite particle. It is also an object of the present invention to provide an apatite particle gene complex, composed of the apatite particle and the gene, combined together, and a method of transfecting the gene with the aid of the apatite particle.

For accomplishing the above object, the present inventors have conducted perseverant researches from variegated aspects. As a result of these researches, the present inventors have found that, if, in forming an apatite particle from inorganic phosphoric acid and calcium ions, magnesium ions are added further, the particle size may be suppressed to a nano-order size, and that, if the resulting apatite particle is used for gene transfection, it is possible to improve the favorable effect of gene transfection into the cell and gene expression in the cell.

The present invention has been brought to completion based on the above finding. That is, an apatite particle according to the present invention is represented by a molecular formula Ca₁₀₋ₓMgₓ(PO₄)₆(OH)₂, where x=1, 2, . . . , 9, or by a chemical formula Ca₈₋ₓMgₓH₂(PO₄)₆, where x=1, 2, . . . , 7, with the particle size being 30 nm to 2500 nm, preferably 50 nm to 1000 nm and more preferably 50 nm to 300 nm.

A method of producing an apatite particle, according to the present invention, produces an apatite particle represented by a molecular formula Ca₁₀₋ₓMgₓ(PO₄)₆(OH)₂, where x=1, 2, . . . , 9, or by a chemical formula Ca₈₋ₓMgₓH₂(PO₄)₆, where x=1, 2, . . . , 7, with the particle size being 30 nm to 2500 nm, preferably 50 nm to 1000 nm and more preferably 50 nm to 300 nm, by incubating a solution containing inorganic phosphoric acid, calcium ions and magnesium ions for a predetermined time duration.

An apatite particle-gene complex, according to the present invention, is such a one in which a specified gene is combined with an apatite particle, which apatite particle is represented by a molecular formula Ca₁₀₋ₓMgₓ(PO₄)₆(OH)₂, where x=1, 2, . . . , 9, or by a chemical formula Ca₈₋ₓMgₓH₂(PO₄)₆, where x=1, 2, . . . , 7, with the particle size being 30 nm to 2500 nm, preferably 50 nm to 1000 nm and more preferably 50 nm to 300 nm.

A method of transfecting a preset gene into a specified cell, according to the present invention, transfects a preset gene into a specified cell by incubating, with said specified cell, an apatite particle-gene complex composed of an apatite particle and said preset gene in which said apatite particle is represented by a molecular formula Ca₁₀₋ₓMgₓ(PO₄)₆(OH)₂, where x=1, 2, . . . , 9, or by a chemical formula Ca₈₋ₓMgₓH₂(PO₄)₆, where x=1, 2, . . . , 7, with the particle size being 30 nm to 2500 nm, preferably 50 nm to 1000 nm and more preferably 50 nm to 300 nm.

Thus, by adding magnesium ions, the particle size of the apatite particle may be suppressed to a nano-order size, whereby it is possible to improve the efficiency in gene transfection into the cell and the efficiency in gene expression in the cell. This has not as yet been reported and has been found out for the first time by the present inventors.

Other objects and specified advantages of the present invention will become more apparent from the following explanation of preferred embodiments thereof especially when read in conjunction with the drawings.

### BRIEF DESCRIPTION OF THE DRAWINGS

FIG. 1 is a graph showing a Fourier transform-infrared spectrum of a conventional apatite particle not containing magnesium ions.

FIG. 2 is a graph showing an X-ray diffraction pattern of a conventional apatite particle not containing magnesium ions.

FIG. 3 is a graph showing changes in turbidity of a particle-dispersed liquid against plural values of the magnesium ion concentration.

FIG. 4 is a graph showing changes in the particle size of the apatite particle against plural values of the magnesium ion concentration.

FIG. 5 shows photos indicating the results of observation of phosphorescent light of a HeLa cell in case a PI-labeled DNA has been transfected into the cell using an apatite particle.

FIG. 6 is a graph showing the status of gene expression in case of transfecting luciferase gene into a HeLa cell using an apatite particle.

FIG. 7 is a graph showing the status of gene expression in case of transfecting luciferase gene into an NIH3T3 cell using an apatite particle.

### BEST MODE FOR CARRYING OUT THE INVENTION

An embodiment of the present invention will now be described in detail with reference to specified experimental results.

Preparation of an Apatite Particle

Initially, classical apatite particles, not containing magnesium ions, were prepared. Specifically, an HBS solution (HEPES Buffered Saline; 140 mM NaCl, 5 mM KCl and 25 mM HEPES, pH: 7.05) containing 0.75 mM Na₂HPO₄.2H₂O was added to with calcium chloride, so that the final concentration was 125 mM, and the resulting product was incubated at room temperature to prepare apatite particles. Then, precipitates of the apatite particles were purified on centrifugation and repeatedly washed with deionized water. The resulting product was then lyophilized.

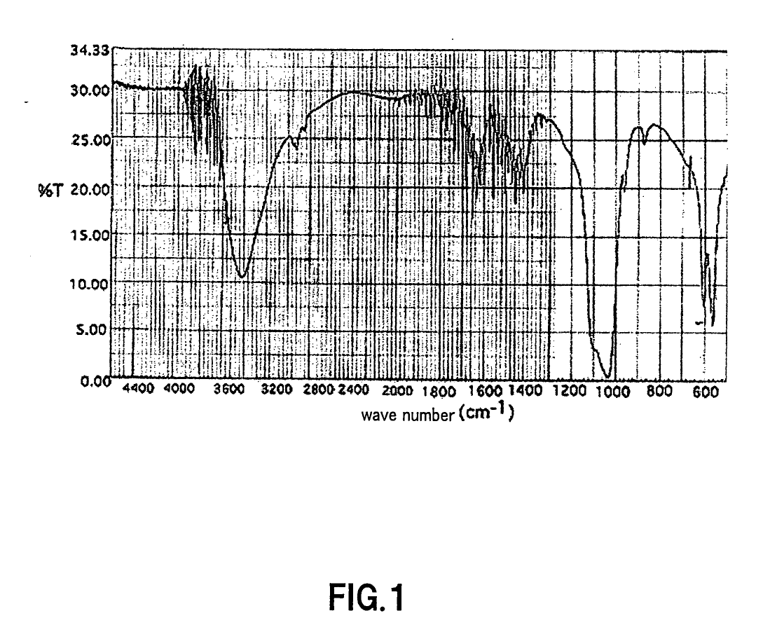

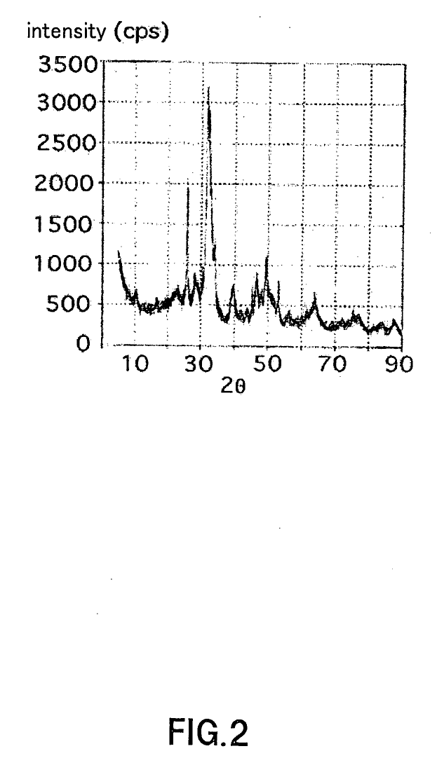

The Fourier transform- infrared spectrum (FT-IR) and the X-ray diffraction pattern of these apatite particles are shown in FIGS. 1 and 2, respectively. The Fourier transform-infrared spectrum was measured using FT-IR-230, manufactured by JASCO Co. Ltd., while the X-ray diffraction pattern was measured using M18XHF-SRA, manufactured by Mac. Sci. Co. Ltd. The infrared spectrum, shown in FIG. 1, indicates that the hydroxyl apatite has been formed. Meanwhile, the peaks at 1000 to 1100 cm⁻¹ and at 550 to 650 cm⁻¹ represent phosphate in the structure. The X-ray pattern, shown in FIG. 2, also shows typical apatitic features.

Then, apatite particles, containing magnesium ions, were prepared. Specifically, these apatite particles were prepared in the same way as above except adding magnesium chloride to the HBS solution to give the final concentration of magnesium chloride of 0, 20, 40, 60, 80, 100, 120 and 140 mM. The apatite particles, obtained on addition of 0, 20, 40, 60, 80, 100, 120 and 140 mM of magnesium chloride, are labeled samples 1, 2, 3, 4, 5, 6, 7, and 8, respectively. The results of elementary analyses of the respective samples are shown in the following Tables 1 and 2. It is noted that Tables 1 and 2 show the weight mass ratio and the molar ratio of Mg, Ca and P in the respective samples, respectively. The amounts of Mg, Ca and P were measured using a Seiko SPS 1500VR atomic absorption spectro-chemical measurement unit, manufactured by SEIKO Co. Ltd.

TABLE 1

| Samples | Mg (%) | Ca (%) | P (%) |
|---|---|---|---|
| Sample 1 | 0.0 | 27.31 | 12.53 |
| Sample 2 | 0.58 | 26.06 | 12.27 |
| Sample 3 | 1.03 | 24.89 | 12.35 |
| Sample 4 | 1.76 | 26.73 | 15.95 |
| Sample 5 | 2.38 | 26.63 | 16.05 |
| Sample 6 | 2.54 | 26.52 | 16.67 |
| Sample 7 | 2.88 | 26.46 | 16.67 |
| Sample 8 | 3.16 | 25.57 | 16.88 |

TABLE 2

| Samples | Mg | Ca | P |
|---|---|---|---|
| Sample 1 | 0.0 | 10.1 | 6 |
| Sample 2 | 0.36 | 9.83 | 6 |
| Sample 3 | 0.64 | 9.39 | 6 |
| Sample 4 | 0.84 | 7.76 | 6 |
| Sample 5 | 1.13 | 7.67 | 6 |
| Sample 6 | 1.16 | 7.37 | 6 |
| Sample 7 | 1.3 | 7.21 | 6 |
| Sample 8 | 1.43 | 7.04 | 6 |

It is seen from Table 1 that, as the concentration of magnesium ions added is increased, the amount of Mg in the apatite particle is increased up to approximately 3% at the maximum, at the same time as the amount of Ca is decreased. However, the amount of P is approximately constant and is approximately 12% and 16% for samples 1 to 3 and for samples 4 to 8, 10 respectively. This indicates that the apatite particles prepared are of one or the other of two types. It may be seen from Table 2 that the samples 1 to 3 are a set of apatite particles with a molecular formula of Ca₁₀₋ₓMgₓ(PO₄)₆(OH)₂, where x=1, 2, . . . , 9, and that the samples 4 to 8 are a set of apatite particles of an OCP (OctaCalcium Phosphate) type, with a molecular formula of Ca₈₋ₓMgₓH₂(PO₄)₆, where x=1, 2, . . . , 7. That is, it has been shown that formation of the OCP type apatite particles may be promoted in the presence of magnesium ions to a higher concentration.

Control of Growth Rate and Size of Particles

Control of the growth rate of the apatite particles by addition of magnesium ions was then scrutinized. It is noted that formation of apatite nuclei and time-dependent growth of particles in an oversaturated solution may be analyzed by measurement of turbidity of a particle-dispersed liquid (see the above Publication 4). So, the turbidity of the particle-dispersed liquid was measured. Specifically, 300 μl of an HBS solution (pH: 7.05), with two-fold concentration, containing 1.5 mM of Na₂HPO₄.2H₂O, were mixed with 300 μl of pure water, containing 250 mM of calcium chloride and 0 to 280 mM of magnesium chloride, were mixed together to generate a particle-dispersed liquid, and changes in turbidity of this particle-dispersed liquid at 320 nm were measured for one to 30 minutes, using SmartSpec™3000, manufactured by Bio-Rad Inc.

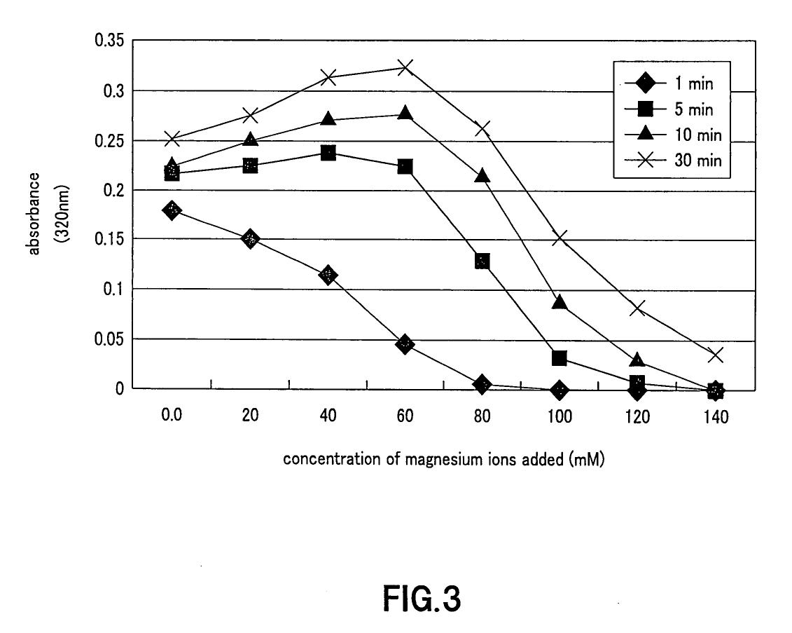

FIG. 3 shows changes in turbidity of the particle-dispersed liquid. It may be seen from FIG. 3 that, when one minute elapsed as from the above mixing, the particle-dispersed liquid was lowered in turbidity with increase in the magnesium ion concentration. This indicates that the growth of particles is suppressed in the presence of magnesium ions. When the incubation was further continued for 5 to 30 minutes, the particle-dispersed liquid was increased and subsequently decreased in turbidity, with increase in the concentration of magnesium ions. This may be explained in such a manner that, by increasing the magnesium ion concentration from 20 to 60 mM, the precipitation reaction is promoted in dependence upon the incubation time, whereby the liquid is increased in turbidity due to the increased number of particles, and that, by further increasing the magnesium ion concentration from 80 to 140 mM, the growth of particles is impeded.

For understanding more clearly the fact that the growth of particles is suppressed, and increase in the particle size is also suppressed, by addition of magnesium ions, changes in the particle size in the course of the growth of particles were observed. Specifically, the average particle size in the stage of growth of particles (1 to 30 minutes) was estimated with a 75 mW Ar laser, using a dynamic spectro-photometer by light scattering ‘Photal’ manufactured by Otsuka Electronics Co. Ltd.

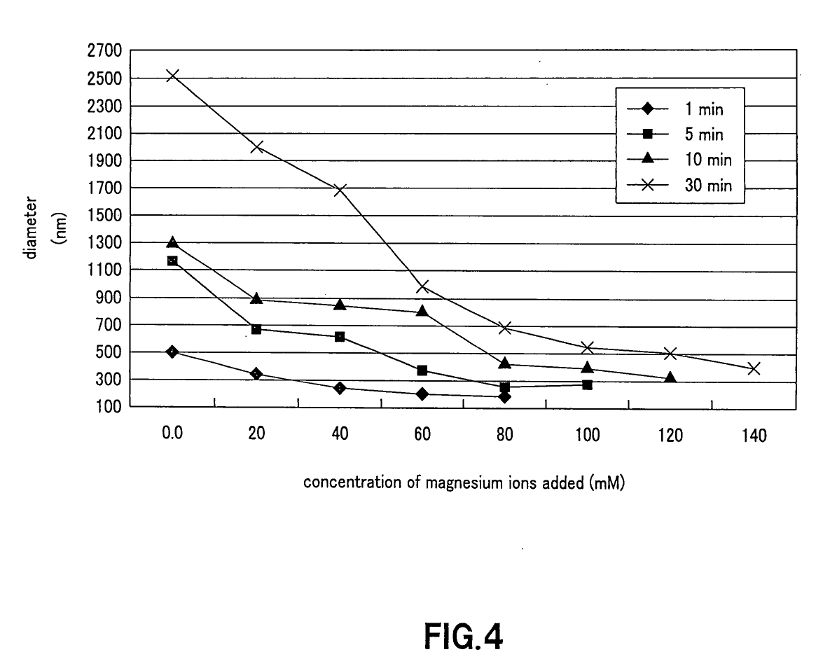

FIG. 4 shows changes in size of the apatite particles. It is seen from FIG. 4 that, within a time interval of from 1 to 30 minutes, as from start of particle formation, the particle size becomes smaller from the micro-order size to the nano-order size, with increase in the magnesium ion concentration.

From FIGS. 3 and 4, definite and reliable prediction may be made on the rate of growth of particles. That is, it may be predicted that, as magnesium ions of higher concentration are taken up by the particles, the rate of growth of particles becomes slower, while the particle size is suppressed to the nano-order size. The effect of magnesium ions on suppression of growth of particles may be explained in such manner that the molecular structure of hydroxyl apatite has become distorted by substitution of magnesium ions for calcium ions in the apatite particles.

Uptake into the Cell of DNA Delivered with Apatite Particles

The particle size represents a crucial factor in gene transfection to the cell. The particle of a smaller size is able to transfer the gene efficiently. However, the particle grows in size promptly. With the particle, increased in size, as a result of precipitous growth, delivery of the gene to the cell and expression of the gene in the cell are obstructed significantly (see FIG. 4). With the apatite particle of the present embodiment, the growth of particles and the particle size may be controlled to a desired level. Thus, the DNA delivery to the cell with the use of the apatite particles was scrutinized.

Specifically, HeLa cells were cultured on a culture medium in a 75 cm² bottle flask under a condition of 5% CO₂ and 37° C. As the culture medium, a DMEM culture medium (Dulbecco Modified Eagle Medium, manufactured by Gibco Co. Ltd.), containing 10% FBS (Fetal Bovine Serum), 50 μg/ml penicillin, 50 μg/ml streptomycin and 100 μg/ml neomycin, was used. One day before the day of DNA transfection, cells from the exponentially growth phase were seeded in a 24-well plate, at a rate of 50,000 cells per well, and cultured in a 50% cell confluency.

Then, 300 μl of an HBS solution of two-fold concentration, containing 1.5 mM Na₂HPO₄.2H₂O (pH: 7.05) were mixed with 300 μl of pure water containing 6 μg of DNA, having 6 μg of PI (phosphorescent probe) as an intercalator (weight ratio of PI to DNA: 1:1), 250 mM of calcium chloriode and 0 to 280 mM of magnesium chloride, were mixed together and incubated for 1 to 30 minutes. A plural number of particle solutions, each being 100 μl, were sampled, and the so sampled particle solutions were added to 1 ml of a culture medium containing the 10% serum. After incubation at 37° C. for four hours, the cells were rinsed with a PBS (Phosphate Buffered Saline) solution containing 5 mM EDTA and phosphorescent light emitted from the samples was checked.

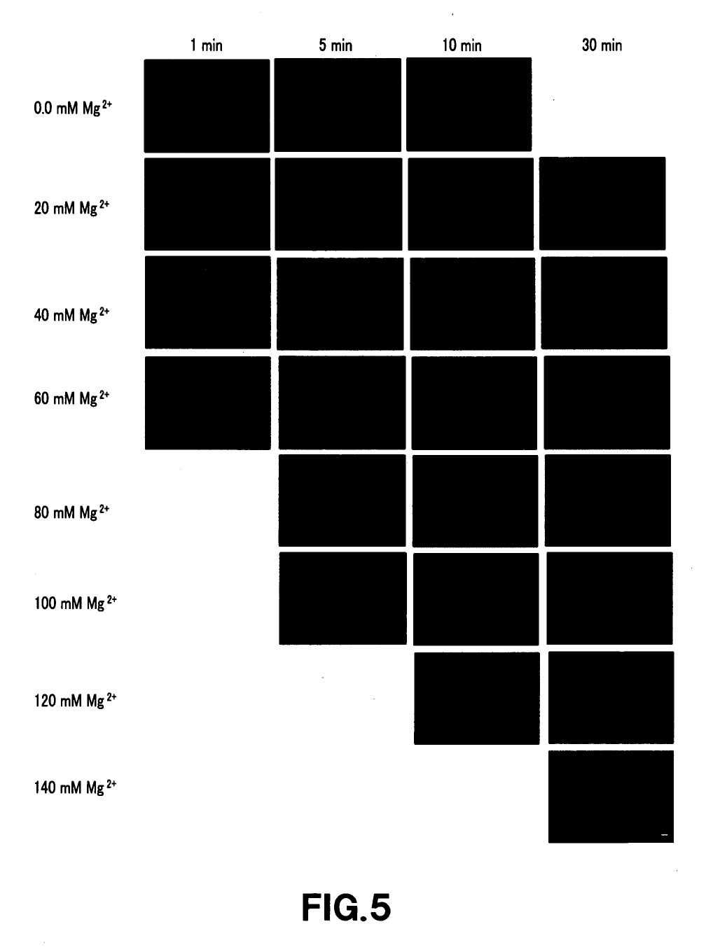

The results of check of phosphorescent light are shown in FIG. 5. The scale bar in FIG. 5 is 50 μm. As may be seen from FIG. 5, if an apatite particle, not added by magnesium ions, is used, DNA uptake into the cell is inefficient. Moreover, as the particle grows in size, with lapse of time, the uptake was decreased to the lowest level. If conversely the apatite particle, added by magnesium ions to the concentration capable of sufficiently suppressing the growth of particles (see FIG. 4), strong phosphorescent light, proper to DNA, labeled with PI, in the inside of the cell, could be observed. That is, it has become apparent that, since the growth of particles has been suppressed due to the magnesium ions added, the DNA-apatite particle complex may be efficiently taken up into the cell by endocytosis. Meanwhile, the reason the DNA uptake into the cell has been lowered with the apatite particle prepared on addition of magnesium ions to higher concentration is presumably such that, with increase in the number of magnesium ions, the apatite particles of the level of occurrence of the precipitation reaction have ceased to be formed.

Expression in the Cell of Genes Delivered Using Apatite Particles

Finally, the state of expression of the luciferase gene in the cell in case the luciferase gene has been transfected into the cell using the apatite particle of the present embodiment was scrutinized.

Specifically, HeLa cells and NIH3T3 cells were each cultured on a culture medium in a 75 cm² bottle flask under a condition of 5% CO² and 37° C. As the culture medium, a DMEM culture medium, containing 10% FBS, 50 μg/ml penicillin, 50 μg/ml streptomycin and 100 μg/ml neomycin, manufactured by Gibco Co. Ltd., was used. One day before the day of DNA transfection, cells from the exponentially growth phase were seeded in a 24-well plate at a rate of 50,000 cells in each well, and were cultured in a 50% cell confluency.

Then, 300 μl of an HBS solution of two-fold concentration containing 1.5 mM Na₂HPO₄.2H₂O (pH: 7.05) were mixed with 300 μl of pure water containing 6 μg of plasmid DNA, containing in turn a luciferase gene pGL3 manufactured by Promega Inc., having 6 μg of PI as an intercalator, with the weight ratio of PI to DNA of 1:1, 250 mM of calcium chloriode and 0 to 280 mM of magnesium chloride, and the resulting product was incubated for 1 to 30 minutes. A plural number of particle solutions, each being 100 μl, were sampled, and the so sampled particle solutions were added to 1 ml of a culture medium containing the 10% serum. After incubation at 37° C. for four hours, the culture medium was exchanged to a fresh culture medium and cultivation was further continued for one day. Subsequently, the state of gene expression in the HeLa cells and in the NIH3T3 cells was checked using a commercial kit manufactured by Promega Inc. and a photon counter (TD-20/20 Luminometer) manufactured by Promega Inc.

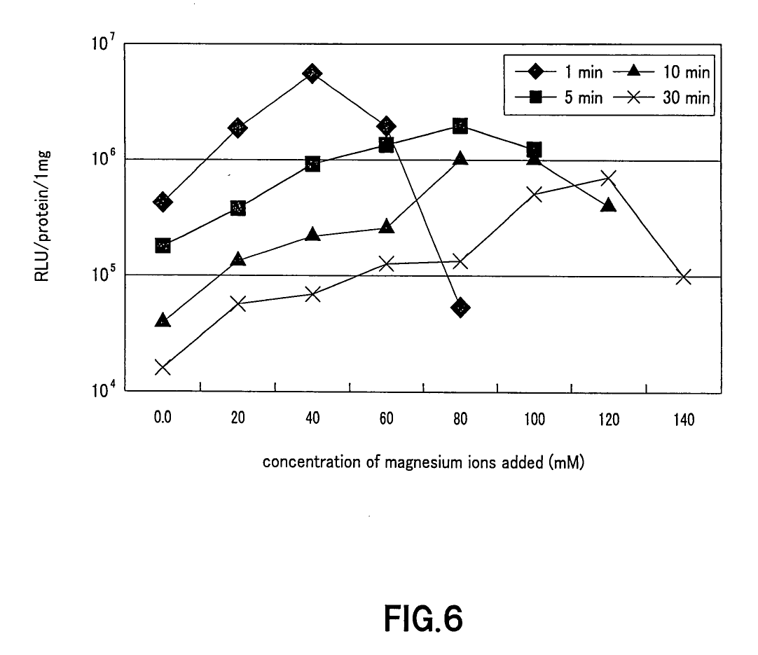

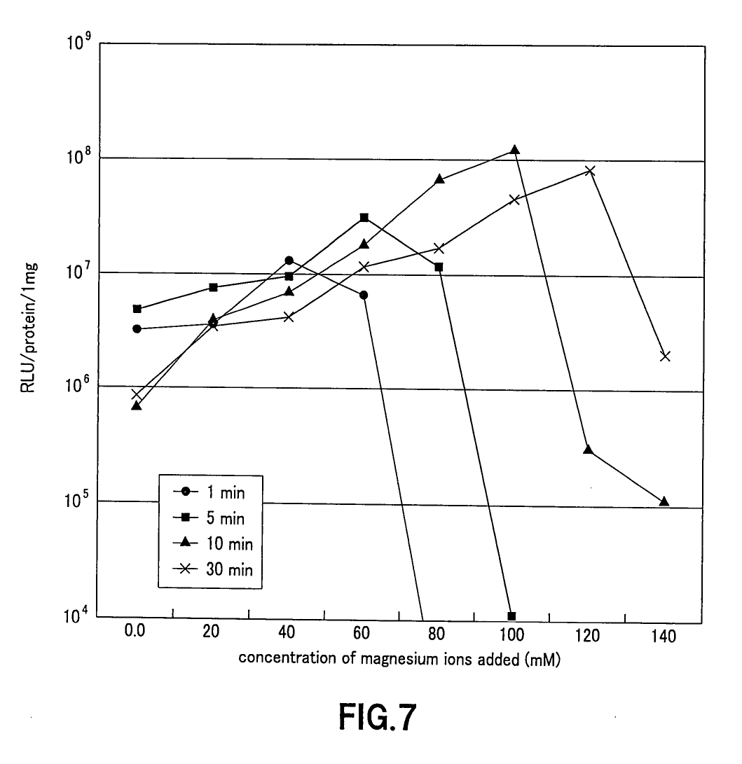

The states of gene expression in the HeLa cell and in the NIH3T3 cell are shown in FIGS. 6 and 7, respectively. In these figures, the efficiency of gene expression in the test for gene transfection, which was carried out thrice, is represented by average light emission per 1 mg of cell protein. It may be seen from FIGS. 6 and 7 that, in case apatite particles, added to with magnesium ions, are used, the gene expression, which is higher at least by 10 to 100 times that in case of using apatite particles, not containing magnesium ions, may be achieved, depending on the concentration of magnesium ions, incubation time or on the sorts of the cells. The reason the gene transfection efficiency is that high is such that, by adding magnesium ions of an optimum concentration, the growth of the apatite particles may effectively be suppressed to suppress the particle size to a nano-order size.

Preferably, the magnesium ion concentration is set in dependence upon the incubation time and on the cell sorts. In the case of NIH3T3 cells, for example, the magnesium concentration is desirably 40 mM, 60 mM, 100 mM and 120 mM, for the incubation time durations of 1 minute, 5 minutes, 10 minutes and 30 minutes, respectively. It is seen from FIG. 4 that, for the magnesium ion concentration of 40 mM and the incubation time of 1 minute, an apatite particle with a particle size of approximately 250 nm is obtained, and that, for the magnesium ion concentrations of 60 mM, 100 mM and 120 mM, with the incubation time durations of 5, 10 and 30 minutes, respectively, an apatite particle with a particle size of approximately 400 nm is obtained. From the perspective of the gene transfection efficiency and the gene expression efficiency, the particle size of 30 nm to 2500 nm is preferred, while the particle size of 50 nm to 1000 nm is more preferred and the particle size of 50 nm to 300 nm is most preferred.

Although preferred embodiments of the present invention have so far been explained with reference to the specified test results, the present invention is not limited to the particular embodiments as explained with reference to the drawings. It will be appreciated that the present invention may encompass various changes or substitutions by equivalents such as may readily be arrived at by those skilled in the art without departing from the scope and the principle of the invention as defined in the claims.

### INDUSTRIAL APPLICABILITY

According to the present invention, described above, the apatite particle with the particle size suppressed to the nano-order size may be produced. Hence, by combining the apatite particle with the gene, gene transfection into the cell and gene expression in the cell may be achieved to high efficiency.

1.-4. (canceled)

5. An apatite particle represented by a molecular formula Ca₁₀₋ₓMgₓ(PO₄)₆(OH)₂, where x=1, 2, . . . , 9, or a chemical formula Ca₈₋ₓMgₓH₂(PO₄)₆, where x=1, 2, . . . , 7, wherein the particle has a size of approximately 30 nm to 2500 nm.

6. The apatite particle of claim 1, wherein the particle size is approximately 50 nm to 1000 nm.

7. The apatite particle of claim 1, wherein the particle size is approximately 50 nm to 300 nm.

8. A method of producing an apatite particle represented by a molecular formula Ca₁₀₋ₓMgₓ(PO₄)₆(OH)₂, where x=1, 2, . . . , 9, or a chemical formula Ca₈₋ₓMgₓH₂(PO₄)₆, where x=1, 2, . . . , 7, wherein the particle has a size of approximately 30 nm to 2500 nm, the method comprising incubating a solution containing inorganic phosphoric acid, calcium ions and magnesium ions for a predetermined time.

9. The method of claim 8, wherein the particle size is approximately 50 nm to 1000 nm.

10. The method of claim 8, wherein the particle size is approximately 50 nm to 300 nm.

11. An apatite particle- gene complex in which a specified gene is combined with an apatite particle, the particle being represented by a molecular formula Ca₁₀₋ₓMgₓ(PO₄)₆(OH)₂, where x=1, 2, . . . 9, or a chemical formula Ca₈₋ₓMgₓH₂(PO₄)₆, where x=1, 2, . . . , 7, wherein the particle has a size of approximately 30 nm to 2500 nm.

12. The apatite particle-gene complex of claim 11, wherein the particle size is approximately 50 nm to 1000 nm.

13. The apatite particle-gene complex of claim 11, wherein the particle size is approximately 50 nm to 300 nm.

14. A method of transfecting a preset gene into a specified cell by incubating, with said specified cell, an apatite particle- gene complex in which a preset gene is combined with an apatite particle represented by a molecular formula Ca₁₀₋ₓMgₓ(PO₄)₆(OH)₂, where x=1, 2, . . . , 9, or by a chemical formula Ca₈₋ₓMgₓH₂(PO₄)₆, where x=1, 2, . . . , 7, wherein the particle has a size of approximately 30 nm to 2500 nm.

15. The method of claim 14, wherein the particle size is approximately 50 nm to 1000 nm.

16. The method of claim 14, wherein the particle size is approximately 50 nm to 300 nm.
